(19) United States
(12) Patent Application Publication
(10) Pub. No.: US 2021/0001078 A1
(43) Pub. Date: Jan. 7, 2021

(54) SENSORY CHAIR

(71) Applicant: Sensory Oasis, Inc., Jackson, MI (US)

(72) Inventor: Kimberly Jayne, Jackson, MI (US)

(21) Appl. No.: 16/919,756

(22) Filed: Jul. 2, 2020

Related U.S. Application Data
(60) Provisional application No. 62/870,108, filed on Jul. 3, 2019.

(51) Int. Cl. A61M 21/02; A47C 7/18; A47C 1/00

(52) U.S. Cl. CPC A61M 21/02; A61M 2021/0022; A47C 1/00; A47C 7/18

(57) ABSTRACT

A sensory chair includes a seat support with a seat cushion having at least one compartment containing a bead-like media, a back support including a back cushion formed at least in party from a viscoelastic foam, and at least one weight attached to the chair, the weight being movable between a non-use position and a use position, wherein the weight in the use position is capable of overlying a portion of the body of a user seated in the chair. Thigh guides may be included to provide additional compression at the user's thighs.

### BACKGROUND OF THE INVENTION

The present invention relates to seating accommodations for students with disorders such as attention deficit disorder (ADD), attention deficit/hyperactivity disorder (ADHD), sensory perception/processing disorder and autism that create difficulty focusing and learning in a traditional classroom seating arrangement.

Students diagnosed with the above noted disorders can have a very difficult time attending to classroom instruction, due to the need to be out of their seat and moving about the room. This creates behavior difficulties due to numerous disruptions through the day. Teachers are often complaining about the time spent dealing with behaviors and constant disruptions, or the need to have to sit with a student one on one to get them to initiate their work.

The student's brain is often in a state of “fight or flight,” such that they are unable to access the frontal lobe of their brain for executive functioning. Instead, they are utilizing their primitive brain due to neurological damage, and or chemical changes, leading to poor sensory modulation. These issues often lead to poor test scores and learning difficulties.

Some success in increasing learning and reducing disruption has been achieved with the use of weighted compression vests, sensory seat cushions, and adult support aids in the classroom. The weighted vests provide support, but can generally only be worn for a limited period, such as 30 minutes at a time. It can also be difficult to get students to comply with putting it on. As students age, they can become uncomfortable with wearing something that makes them look different. Known sensory seat cushions generally include an inflatable plastic cushion with a textured surface. These cushions may also provide some good support for students, but can often become a distraction or a disadvantage because they are not attached to anything, are often over or under inflated, and can get punctured and rendered unusable.

Another known tool for this type of student is a bean bag chair. These chairs may also help with calming, but do not offer structured seating that pulls up to a table or desk, are big and bulky, and take up significant space in already crowded classrooms.

### SUMMARY OF THE INVENTION

The present invention provides a sensory chair as a solution for the needs of students that includes a seat support including a seat cushion having at least one compartment containing a bead-like media, a back support including a back cushion formed at least in part from a viscoelastic memory foam, and at least one weight attached to the chair, the weight being movable between a non-use position and a use position, wherein the weight in the use position is capable of overlying a portion of the body of a user seated in the chair. The back cushion provides compression via the memory foam cushion, by the students body pushing against the cushion. The memory foam forms to the body and yet retains its shape after the student exits the chair to enable more than one student to use the chair without making cumbersome adjustments to the chair to accommodate different sizes and shapes. The weights provide additional input to the proprioceptors that receive information to help the body know where it is at in space, leading to decreased falling out of classroom chairs, and the need to move about the room. By putting the student in a calm state, the student can access their executive functions and increase their learning potential. Notably, testing of children in a controlled environment utilizing a chair according to the present embodiment has shown up to ten times increased time on task as compared to standard classroom seating.

In one embodiment, the seat cushion includes a plurality of the compartments, with each of the compartments filled with a bead-like media. The compartments may be positioned adjacent to the upper surface of the seat cushion.

In another embodiment, the back cushion has a forward surface facing in the direction of a user seated on the seat support, and the forward surface is concave to at least partially surround the user. This concave portion may be formed from the viscoelastic foam (i.e., “memory foam”). The viscoelastic foam may have a thickness between 1.5 inches and 4 inches.

The chair may include armrest portions extending upwardly on opposing lateral sides of the seat support, and the armrest portions may each be covered with a thigh guide extending inwardly from each of the armrest portions to engage the thighs of a user. Each of the thigh guides include at least a portion made from viscoelastic foam.

In one embodiment, at least one of the weights is attached to the back support and at least one is attached to one of the armrest portions. The weights may each include an adjustable length strap portion and a weighted portion attached to the strap portion. The weighted portion is covered with a cover material, and each weight may be interchangeable for another weight having a different cover material. The weights attached to the armrest portions are capable of being moved to a position overlying the thighs of a user seated in the chair, and the weights attached to the back support are capable of being moved to a position wherein each overlies one of the user's shoulders.

These and other objects, advantages, and features of the invention will be more fully understood and appreciated by reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### BRIEF DESCRIPTION OF THE DRAWINGS

FIG. 1 is a front view of the sensory chair according to one embodiment;

FIG. 2 is a front perspective view thereof;

FIG. 3 is a perspective view thereof with a representative person seated in the chair;

FIG. 4 is a left perspective view of a partially assembled chair;

FIG. 5 is a front view of a partially assembled chair;

FIG. 6 is a right perspective view of a partially assembled chair;

FIG. 7 is a left side, section view of a partially assembled chair;

FIG. 8 is a bottom view of a partially assembled chair;

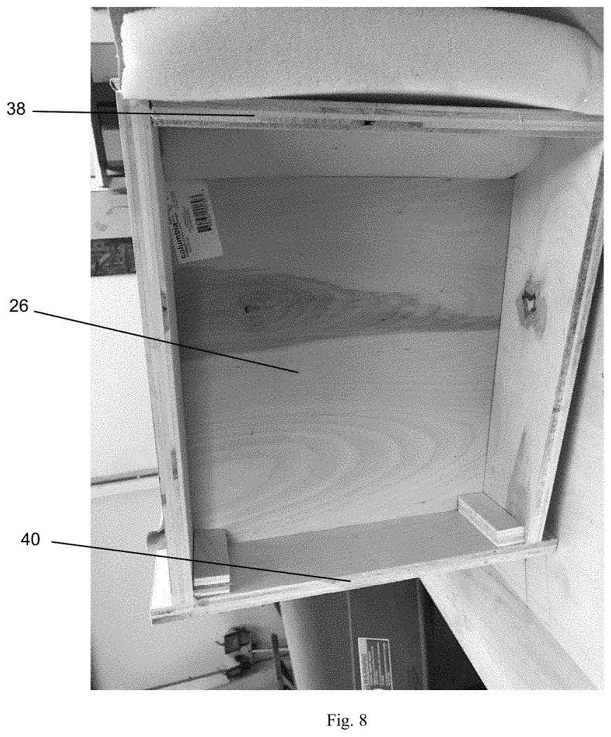

FIG. 9 is a rear view of a partially assembled chair.

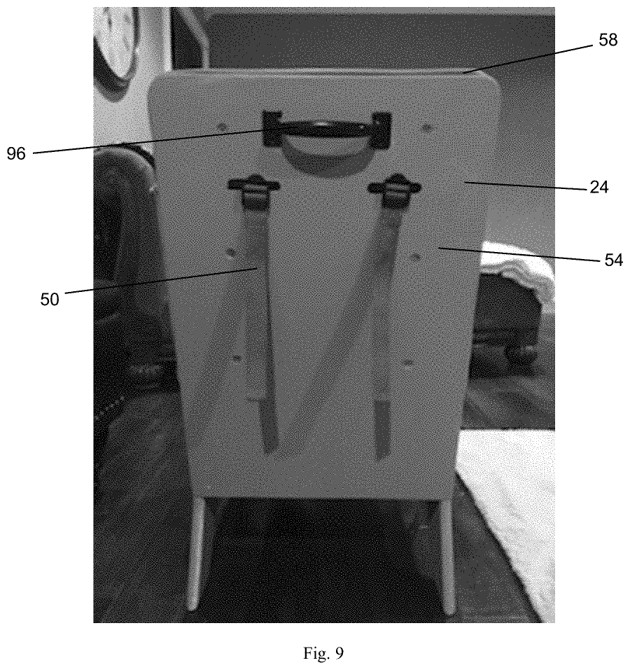

### DESCRIPTION OF THE CURRENT EMBODIMENT

I. Overview

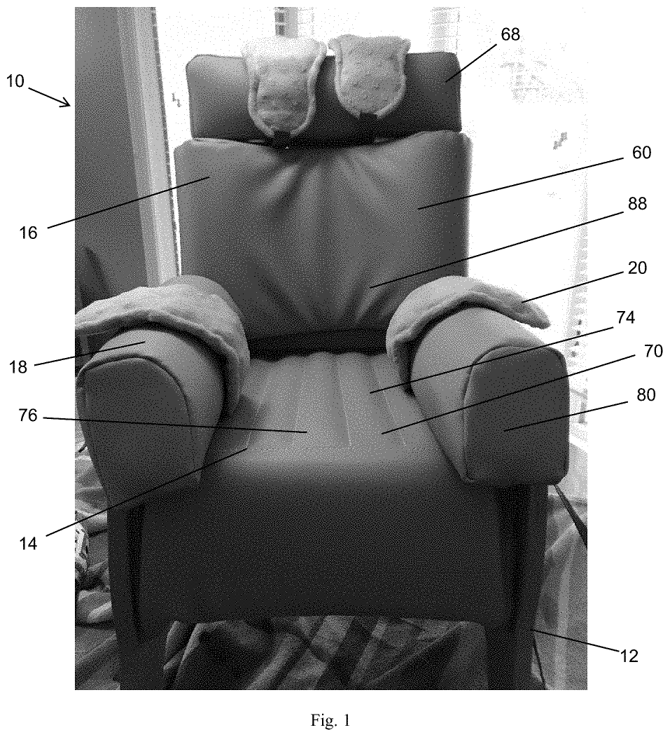

A chair for supporting users with disorders such as attention deficit disorder (ADD), attention deficit/hyperactivity disorder (ADHD), sensory perception/processing disorder and autism, generally referred to as a “sensory chair” is shown in FIG. 1 and generally designated 10. In one embodiment, the chair 10 includes a ground-engaging base 12, a seat support 14, a back support 16, and a pair of armrests 18 that may additionally act as thigh supports. The chair 10, seat support 14, back support 16 and armrests 18 may each be provided with a type and degree of cushioning designed to engage the user with a degree of compression that facilitates a calming output. The chair 10 may additionally include attached weighted elements 20 that can be manipulated by the user to provide weight to one or more portions of the user's body.

II. Structure

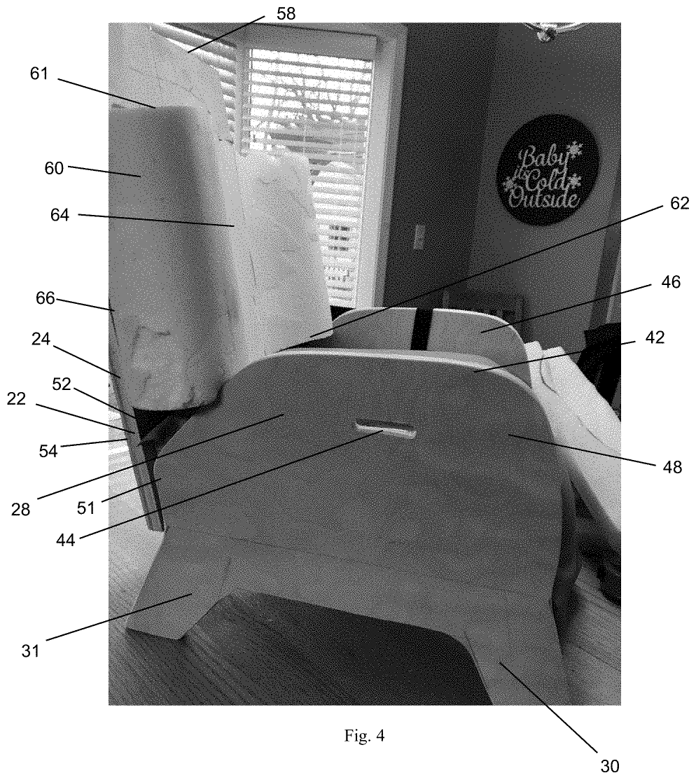

With reference to FIGS. 4-6, the chair 10 includes an underlying support structure or frame 22. The frame 22 is shown in one embodiment as being fabricated from wood. The wooden frame 22 may be formed, for example, by cutting and assembling a series of wooden pieces to form a frame 22 of desired size and shape. In an alternative embodiment, the frame 22 can be fabricated from one or more of a variety of materials such as wood, plastics and metals.

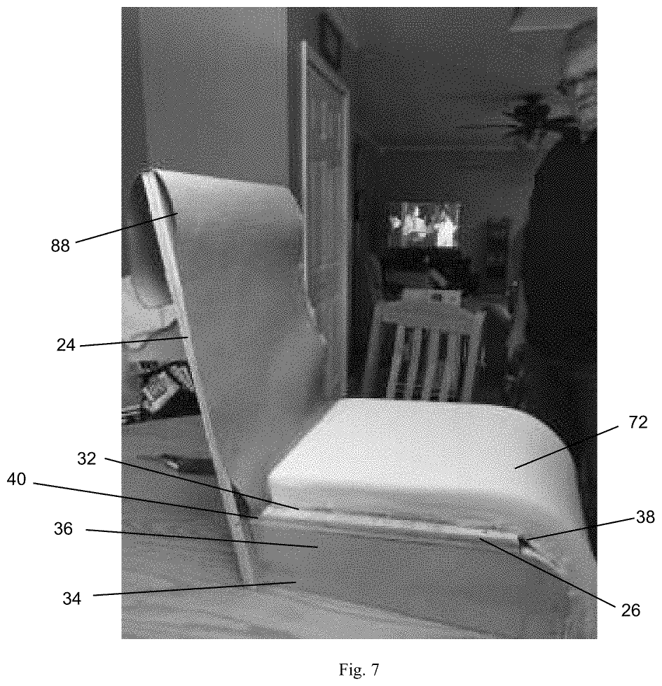

In the illustrated embodiment, the frame 22 includes a back frame 24, a seat frame 26, a pair of armrest frames 28 and a base frame 30. The base frame 30 forms the underlying structure for the ground engaging base 12, and in the illustrated embodiment includes four legs 31 that extend from the floor to the seat frame 26. The seat frame 26 (shown in FIG. 7, which is a portion of the chair 10 with the armrests removed) is a generally horizontal, and generally flat support element, having an upper surface 32, a lower surface 34 opposite the upper surface 32, left and right lateral sides 36, a front edge 38 and a rear edge 40. In one embodiment, the armrest frames 28 are positioned on the lateral sides 36 such that the armrest frames 28 extend upwardly from the upper surface 32 at the lateral sides 36. Each armrest 28 includes a rounded upper edge 42, and in one embodiment, each armrest includes a slot 44 extending laterally through the armrest 28 from an interior surface 46 to an exterior surface 48 for receiving a weight attachment strap 50 as discussed in more detail below. Although the positioning of the slots may be moved from application to application, the slots 44 are generally spaced above the upper surface 32 of the seat frame 26. In one embodiment, each armrest frame 28 is formed as a single unitary piece with an associated one of the base frames 30; however, in another embodiment the armrest frames 28 and the base frames 30 may be separate elements. Optionally, wheels may be added to the back legs 31 of the chair 10 to enable the chair 10 to be tipped chair back and rolled to a new area. When the chair 10 is in an upright position, the wheels will not make contact with the floor for safety reasons.

As shown, the back frame 24 forms a rigid structure for a backrest for the chair 10. The back frame 24 extends upwardly from the upper edge 32 of the seat frame 26 at the rear edge 40 of the seat frame 26. In the illustrated embodiment, the back frame 24 is attached to a rear edge 51 of the armrests 28; however, the back frame 24 may alternatively be attached to the base frame 30 or the seat frame 26. The back frame 24 includes a front surface 52 facing the seat frame 26 and a rear surface 54 opposite the front surface 52. The back frame 24 further includes a top edge 58 that defines a back support height. In one embodiment, the back support height is sufficient to extend above the user's shoulders and behind the head of a user to form a headrest portion 57. In one embodiment, the back frame 24 includes a pair of slots 56 extending through the back frame 24 from the front surface 52 to the rear surface 54. The slots 56 are located on opposite sides of a generally vertical centerline through the back frame 24 at a height that is spaced below the top edge 58 of the back frame 24 at a position that is approximately aligned with the upper surface of a user's shoulders. In one embodiment, the slots 56 are advantageously positioned in the upper half of the backrest height, and in a more particular embodiment the slots 56 are positioned at about the bottom edge of the top ⅓ of the backrest height to form a bottom edge of the headrest portion 57.

Each of the back frame 24, the seat frame 26, and armrest frames 28 may be covered with a desired cushioning material to provide the user with a degree of compression. As shown, at least a portion of the front surface 52 of the back frame 54 is covered with a back cushion 60. In one embodiment, the back cushion 60 includes a bottom edge 62 proximate to the upper edge 32 of the seat frame 26, a top edge 61 generally aligned with the user's shoulders, a front surface 64 facing the user and a rear surface 66 abutting the back frame 24. In one embodiment, the top edge 61 is positioned adjacent to the slots 56. In the illustrated embodiment, the back cushion 60 has a concave front surface 64 to surround a portion of the user's scapular, spinal and latissimus muscles. In one embodiment, the concave front surface 64 has a 2¼″ radius of curvature for creating a desired degree of compression for the user. The back cushion 60 may be formed with memory foam to provide the user with a compression feel, and in one embodiment the back cushion 60 is formed from 1.5″-4.0″ thick, high density, viscoelastic polyurethane foam, commonly referred to as memory foam. In another embodiment, the foam may be a gel visco foam.

Referring to FIGS. 1-3, in one embodiment, the headrest portion 57 (i.e., the portion of the back support 16 above the user's shoulders and generally above the slots 56) is covered with a headrest cushion 68, which may be a standard polyurethane foam or a memory foam of about 1″-3″ thickness. In one optional embodiment, a Bluetooth speaker is installed in the headrest portion 57 to allow a teacher or aide to use a compatible device to communicates with the speaker for individual opportunities for instruction.

The seat frame 26 is covered with a seat cushion 70. In one embodiment, the seat cushion 70 is comprised of a cushion material that is different from that of the back cushion 60 and headrest cushion 68 (such that the seat frame 26, back frame 24 and headrest frame portion 57 are covered with three different materials, respectively). The seat cushion 70 may have multiple layers, including a first layer of utility seating foam 72 (shown in FIG. 7), and a second, upper layer that is a beaded seat cushion 74 placed over the first layer 72. As shown in FIG. 1, the beaded cushion 74 includes 1″-2″ sewn chambers 76 filled ¼-¾ of their volume with poly beads or another bean-bag-style filler to allow for movement and sensory input. The chambers 76 extend parallel to each other and in a front-to-back direction on the seat cushion 70. In one embodiment, each of the cushions may be covered or wrapped with a cover material 88, such as vinyl, leather, cloth or another upholstery material. The chambers 76 may be formed directly in the cover material 88. The upholstery for the seat 14 and back 16 may attach the cushions to the chair 10.

The armrest frames 28 may each be covered with a cushion cover 80, which forms a thigh guide for the user. In one embodiment, each thigh guide 80 is generally cylindrical, and is positioned inboard of the armrest frames 28 and above the seat cushion 70 such that they engage a user's thighs. The thigh guides 80 may be formed from memory foam of 1″-4″ thickness for compression against the thighs. In one embodiment, additional trunk supports may be provided with the chair 10 to be placed inside the thigh guides 80 to provide an added compression input on the user. The trunk supports may be attached in a hinged manner such that they are able to swing out of the way (in a manner similar to the weights 20) when not used. The trunk supports may be 1″-3″ of memory foam cushion. In one embodiment, both the thigh guides 80 and the optional trunk supports are offered a range of thicknesses in the foam size depending on how much compression is needed. The trunk supports may be added as an option if a user desires additional compression beyond the combination of the back cushion 60 and thigh guides 80. In another embodiment, the back cushion 60 may have a flat front surface and the trunk supports or thigh guides may wrap around the user's back to provide additional or more direct contact through the user's trunk.

As noted, additional thicknesses and sizes of thigh guides 80 may be used, depending on the size of the user and the intended degree of compression. In one embodiment, the chair 10 may be provided with interchangeable sizes of thigh guides 80, each for use with a different size and or type of user. In another embodiment, the chair 10 may include an adjustable thigh guide 80 that is capable of moving in and out to provide compression as desired. The combination of the seat cushion 70, the back cushion 60 and the thigh guides 80 act to support the user with both compression via the memory foam back 60 and thigh guides 80 and tactile feedback via the beaded seat chambers 76.

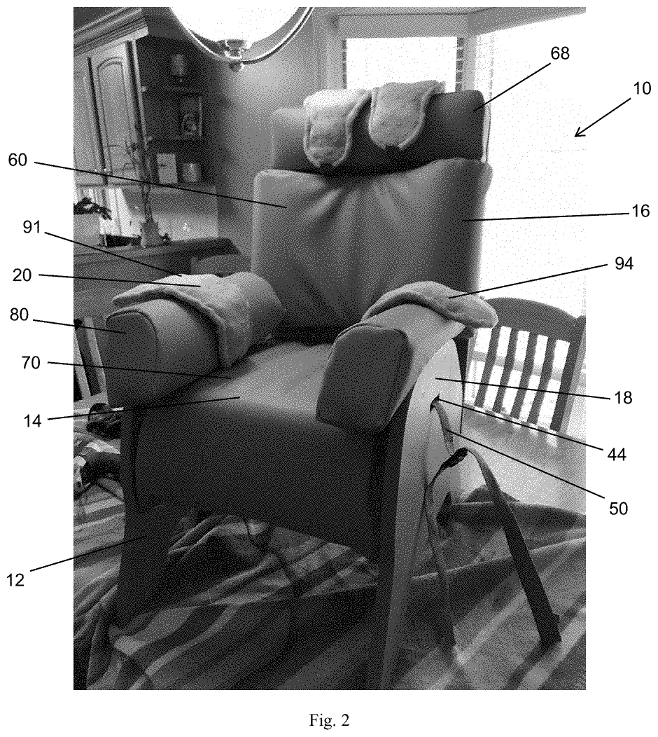

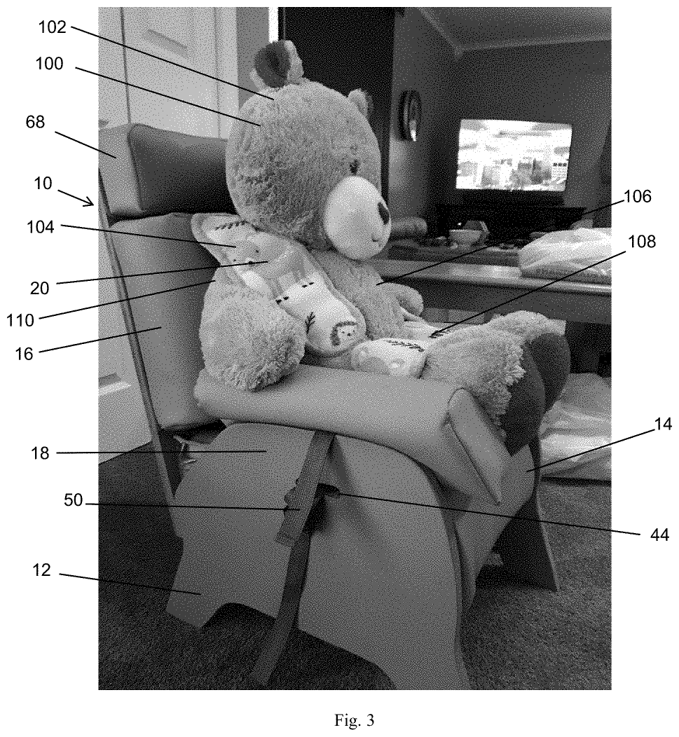

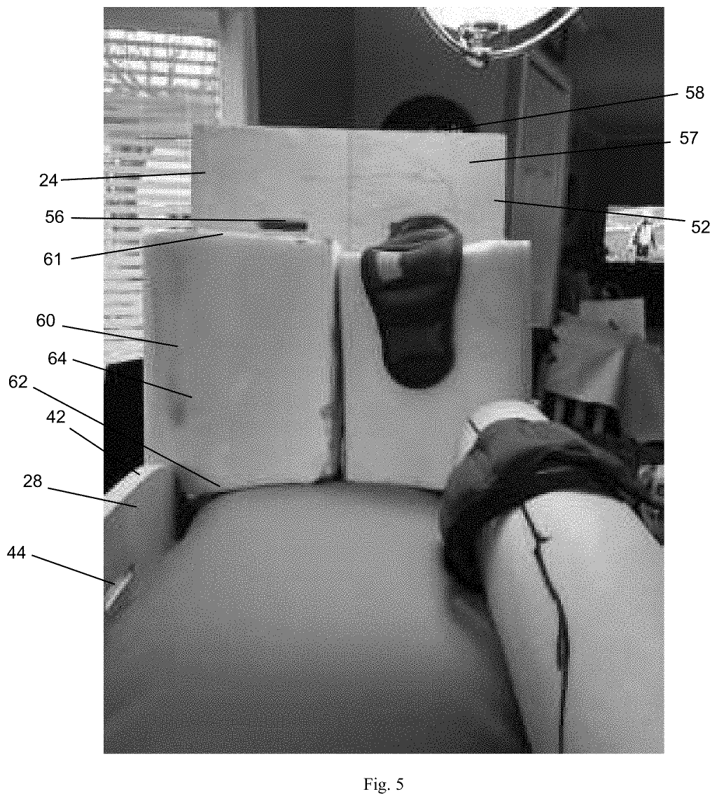

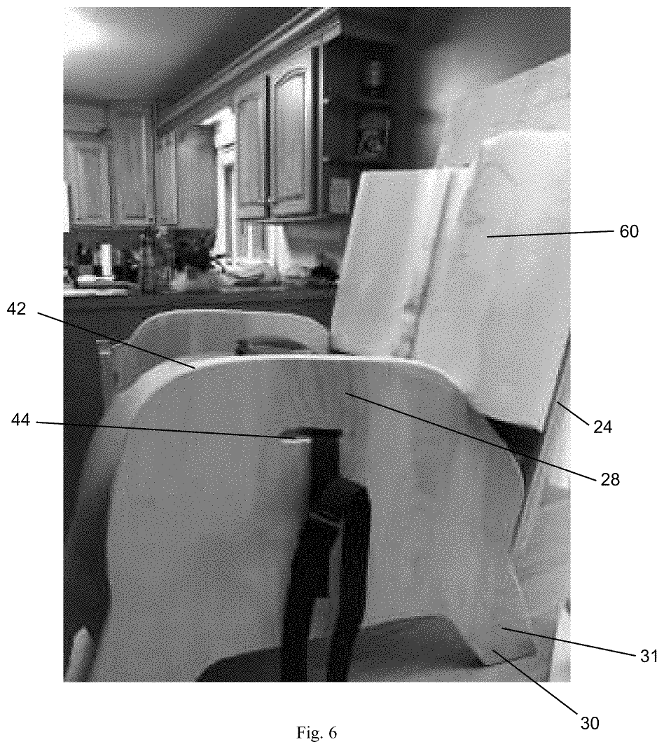

As noted above, the openings or slots in the armrests 44 and at the shoulders 56 are designed for attachment of weights that can be placed on the user's shoulders and thighs. As shown in FIGS. 5 and 6, each weight 20 may include a strap portion 50 and a weight portion 94. The strap enables adjustability and may extend through one of the slots 44 or 56 to attach the weight 20 to the chair 10. Straps 50 for two or shoulder weights 20 may extend through the shoulder slots 56 between the headrest cushion 68 and the back cushion 60, forming over the shoulder length adjustable weights that attach to the chair 10. Straps 50 for two thigh weights 20 may extend through the armrest slots 44, forming over the thigh adjustable length weights 20. The weights 20 may each be individually movable between a non-use position shown in FIGS. 1 and 2 and a use position shown in FIG. 3. In one embodiment, the combined weight of all weights 20 comprises approximately 10-15% of the client's body weight, which is known as a best practice for providing proprioceptive sensory input to calm the body's natural “fight or flight” response in the case of user's with the noted disorders.

In one embodiment, the weights 20 are covered with material 91 that is appealing to the client's sensory needs, such as silky, soft or textured material that appeals to the user's needs for calming and focus. In one embodiment a chair 10 may be provided with a variety of weights 20 of different sizes and coverings such that each weight 20 can be interchanged with another weight to meet the particular needs of the individual user.

Optionally, the chair may include a handle 96 on the back support 16 for transportability. Although not shown, the chair 10 may be combined with a foot rest that has an exterior frame with pressure or tension resistant materials for the user to push against for proprioceptive input cover the top. This footrest may be made to be flat or at an incline of up to 45 degrees. This could be height adjustable to accommodate a client up to 5″. In another embodiment, one or more elements of the chair frame, such as the back frame 24, seat frame 26 and armrest frames 28 may be provided with a degree of adjustability. For example, the back frame 24 may adjustable to recline with respect to the seat frame 26 and the armrest frames may be independently adjustable with respect to the seat 26 and back 24 frames.

Throughout this description, directional terms, such as “vertical,” “horizontal,” “top,” “bottom,” “upper,” “lower,” “inner,” “inwardly,” “outer” and “outwardly,” may be used to assist in describing the invention based on the orientation of the embodiments shown in the illustrations. The use of directional terms should not be interpreted to limit the invention to any specific orientation(s).

III. Operation

Operation and use of the chair 10 is described primarily in connection with FIG. 3, which shows a sample user 100 seated in the chair 10. A typical user includes a head 102, shoulders 104, torso 106, thighs 108 and back 110. When a user 100 is seated in the chair 10, the user's back 110 generally engages the back cushion 60, the user's head 102 is positioned adjacent the headrest cushion 68 and the user's thighs 108 are positioned between the thigh guides 80. The concave front surface 64 of the memory foam back cushion 60 surrounds a portion of the user's scapular, spinal and latissimus muscles within the user's back 110 to provide a compressive input to the user 100. Similarly, the thigh guides 80 may provide the user 100 with compression at the user's thighs 108. The compression is known to provide a regulatory effect on sensory processing, reducing the effect of the stimuli, and helping to calm and soothe an overstimulated nervous system and providing a safe and protected feeling. The compression cushioning can replicate a hug to increase the calming effects on the user's nervous system.

The seat cushion 70 provides the user's backside with a different type of input. In particular, the seat cushion 70 is filled with beads, such as polymeric beads, to provide tactile feedback to the user that is known to decrease the need to fidget or move about. Those skilled in the art will appreciate that it may be advantageous to include the beads in only one cushion, as it may be too stimulating or have an alerting effect to use a tactile bead in additional cushions. In one embodiment, a user will have the option to choose a beaded tactile feedback cushion, or a flat foam cushion seat, depending on their need for the appropriate type of input.

The user may choose to use one or more of any of the weights 20 attached to the chair 10. Initially, the user may select a desired set of the weights 20 for attachment to the chair 10 and use with the chair 10, including a set of weights 20 with a desired cover material 91 and of a desired weight. For example, the chair 10 may be provided with multiple sets of weights 20 separate from the chair 10, and the user may select a desired set of those weights 20 (which may all be the same weight and material or varied weights and material) and then attach the desired set of weights 20 to the chair 10 by extending the adjustable strap 50 through one of the armrest slots 44 or one of the backrest slots 56, and adjusting the straps to desired lengths. In one embodiment, all of the weights 20 may initially be positioned in the non-use position as shown in FIGS. 1 and 2. At the user's option, one or more of the weights 20 may be moved to the use position as shown in FIG. 3. In this use position, each of the shoulder weights 20 are positioned on one of the user's shoulders and a portion of the user's chest, and the thigh weights are positioned across the user's thighs 108. The user can move the weights 20 between the use and the non-use positions as desired.

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular.

The embodiments of the invention in which an exclusive property or privilege is claimed are defined as follows:

1. A sensory chair comprising:
a base;
a seat support on the base, the seat support including a seat cushion having at least one compartment containing a bead-like media;
a back support extending upwardly from the seat support, the back support including a back cushion, wherein at least a portion of the back cushion is formed from a viscoelastic foam; and
at least one weight attached to the chair, the weight being movable between a non-use position and a use position, wherein the weight in the use position is capable of overlying a portion of the body of a user seated in the chair.

2. The sensory chair of claim 1, wherein the seat cushion includes a plurality of the compartments, each of the compartments filled with a bead-like media.

3. The sensory chair of claim 2 wherein the seat cushion includes an upper surface capable of engaging a user, and wherein the compartments are positioned adjacent to the upper surface.

4. The sensory chair of claim 1 wherein the back cushion has a forward surface facing in the direction of a user seated on the seat support, and wherein the forward surface is concave to at least partially surround the user.

5. The sensory chair of claim 4 wherein the portion of the back cushion formed from the viscoelastic foam includes the forward surface.

6. The sensory chair of claim 1 wherein the back cushion includes a thickness defined in the direction extending toward the back of a user, and wherein the portion of the back cushion formed from the viscoelastic foam has a thickness between 1.5 inches and 4 inches.

7. The sensory chair of claim 1 wherein at least one of the weights is attached to the back support.

8. The sensory chair of claim 1 wherein the back support has a back support height extending upwardly from the seat support to a top edge, wherein the back cushion extends to a top edge spaced below the top edge of the back support.

9. The sensory chair of claim 8 wherein the at least one of the weights attached to the back support is positioned above the top edge of the back cushion.

10. The sensory chair of claim 9 wherein the weights each include an adjustable length strap portion and a weighted portion attached to the strap portion.

11. The sensory chair of claim 10 wherein the weighted portion is covered with a cover material.

12. The sensory chair of claim 11 including multiple interchangeable weights, each of said multiple weights having a different cover material.

13. The sensory chair of claim 1 including armrest portions extending upwardly on opposing lateral sides of the seat support.

14. The sensory chair of claim 13 wherein the chair includes thigh guides extending inwardly from each of the armrest portions to engage the thighs of a user.

15. The sensory chair of claim 14 wherein each of the thigh guides include at least a portion made from viscoelastic foam.

16. The sensory chair claim 15 wherein at least a second one of the weights is attached to one of the armrest portions.

17. The sensory chair of claim 16 including two of the weights attached to the respective opposing armrest portions and two of the weights attached to the back support.

18. The sensory chair of claim 17 wherein the weights attached to the armrest portions are capable of being moved to a position overlying the thighs of a user seated in the chair.

19. The sensory chair of claim 18 wherein the weights attached to the back support are capable of being moved to a position wherein each overlies one of the user's shoulders.

20. A chair adapted for users with attention or sensory related disorders, comprising:
a seat support including a lower cushion layer and an upper cushion layer, the lower cushion layer formed from a resilient material, and the upper cushion layer layered over the lower cushion layer and having at least one compartment containing a bead-like media contained in the compartment, the upper cushion layer forming an upper surface of the seat support for engaging a user seated on the seat support;
a back support extending upwardly from the seat support, the back support including a back cushion, wherein at least a portion of the back cushion is formed from a viscoelastic foam, the viscoelastic foam having a concave forward surface facing the user; and
at least one weight attached to the chair, the weight being movable between a non-use position and a use position, wherein the weight in the use position is capable of overlying a portion of the body of a user seated in the chair.

21. A sensory chair comprising:
a base;
a seat support on the base, the seat support including a seat cushion having a plurality of separate compartments, each containing a bead-like media;
a back support extending upwardly from the seat support, the back support including a back cushion, wherein at least a portion of the back cushion is formed from a viscoelastic foam, the back cushion having a central region and lateral sides on either side of the central region, the lateral sides being thicker than the central region to form a concave forward surface of the back support;
a pair of spaced apart thigh guides, one on each lateral side of the seat support, the thigh guides formed of a cushion material and adapted to engage and compress the thighs of a user seated on the seat support; and
at least one weight attached to the chair, the weight being movable between a non-use position and a use position, wherein the weight in the use position is capable of overlying a portion of the body of a user seated in the chair.
